(19) United States
(12) Patent Application Publication
(10) Pub. No.: US 2007/0258918 A1
(43) Pub. Date: Nov. 8, 2007

(54) High DS cationic polygalactomannan for hair conditioner products

(76) Inventor: Jashawant J. Modi, Hockessin, DE (US)

Correspondence Address: Hercules Incorporated;Hercules Plaza, 1313 N. Market Street, Wilmington, DE, 19894-0001, US

(21) Appl. No.: 11/799,535

(22) Filed: May 2, 2007

Related U.S. Application Data
(60) Provisional application No. 60/796,762, filed on May 2, 2006.

(51) Int. Cl. A61K 8/73

(52) U.S. Cl. 424/59; 424/70.17; 424/70.13; 424/70.122

(57) ABSTRACT

A hair conditioner composition is provided with a) from about 1 to about 99 wt % of water, b) at least about 0.05 wt % of a cationic polymer wherein the cationic polymer has a mean average molecular weight (Mw) from about 2,000 to about 10,000,000 Dalton, and the cationic polymer has a cationic degree of substitution (DS) greater than 0.25 to about 3.0, and c) at least one hair conditioner active ingredient, wherein the active hair conditioner ingredient is selected from the group consisting of perfumes, emollients, moisturizing agents, humectants, sunscreen actives, beauty aids, anti-oxidant agent, antimicrobial agent, additional conditioning agent, depositing agent, tanning agent, rejuvenating agent, medicaments agent, and mixtures thereof.

### RELATED APPLICATIONS

This application claims the benefit of U.S. Provisional Application Ser. No. 60/796,762, filed on May 2, 2006, which is incorporated herein by reference in its entirety.

### FIELD OF INVENTION

The present invention relates to hair conditioner compositions. More specifically, this invention relates to a hair conditioner composition containing a cationic polysaccharide where the hair conditioner composition provides an increased ease in combing hair.

### BACKGROUND OF THE INVENTION

In recent years, there has been a need to have a cationic polysaccharide which can provide higher level of hair detangling i.e. hair easier to comb than the commercially available cationic polysaccharides. This need has arise from the high growth rate in Far Eastern countries such as India, China, and Indonesia where people, especially women, desire to have heavy conditioning of hair.

Cationically derivatized cellulosics and polygalactomannan derivatives are used as a conditioning agent for hair and skin. Cellulosics and polygalactomannan polymers without a cationic group(s) are not used as conditioning agents but rather are used only as rheology modifiers. Most of the commercial cationic polygalactomannans, such as cationic guar products, have cationic degree of substitution (DS) of less than 0.20. Because of manufacturing process related issues, only cationic guar with DS of less than 0.2 is commercially available from raw material suppliers, such as Hercules Incorporated, which markets its products under the trademark N-Hance® cationic guar derivatives, and Rhodia Corporation which markets its products under the trademarks Jaguar® natural guar derivatives and Jaguar® Excel guar derivatives. In addition, because of the low DS of the commercial polymers, they are less soluble in water and, therefore, also impact viscosity of the hair conditioners if attempts are made to use higher levels. There are several references that disclose the use of cationic polygalactomannans in personal care products.

U.S. Pat. No. 6,930,078, incorporated herein by reference in its entirety, discloses a shampoo containing a cationic guar derivative having a high degree of substitution.

US Patent Application publication 2003/0108507 A1 discloses a hair conditioning shampoo composition of surfactants and cationic polymers.

US Patent Application publication 2004/0157754 A1 discloses a shampoo composition with a conditioning agent and a cationic polymer with a high charge density.

US Patent Application publication 2003/0202952 A1 discloses a shampoo composition with an anti-dandruff agent and a cationic polysaccharide.

US Patent Application publication 2003/0215522 A1 discloses a personal care composition and a zinc anti-dandruff agent and a cationic polymer.

A need still exists for hair conditioning compositions containing cationic polygalactomannans wherein the composition provides better combing performance as compared to previous compositions.

### SUMMARY OF THE INVENTION

The present invention is directed to a hair conditioner composition consisting essentially of from about 1 to about 99 wt % of water, a lower limit amount of 0.05 wt % of a cationic polymer that has a weight average molecular weight (Mw) of from about 2,000 to about 10,000,000 Dalton, and a cationic degree of substitution (DS) with a lower limit of 0.25 and an upper limit of 3.0, and at least one hair conditioner active ingredient. The hair conditioner composition is substantially surfactant-free and provides ease of combing the hair.

The term “substantially surfactant-free” as used herein shall mean a hair conditioner composition containing less than 1% by weight of a surfactant which is to provide cleaning performance to a composition. Preferably the “substantially surfactant-free” hair conditioner composition contains less than 0.5% by weight of a surfactant, more preferably less than 0.1% by weight of a surfactant.

Examples of the active hair conditioner ingredient is selected from the group consisting of perfumes, emollients, moisturizing agents, humectants, sunscreen actives, beauty aids, anti-oxidant agent, antimicrobial agent, additional conditioning agent, depositing agent, tanning agent, rejuvenating agent, medicaments, colorant, anti-static agent, and mixtures thereof.

### DETAILED DESCRIPTION OF THE INVENTION

It has been surprisingly found that using of cationic guar polymers having a DS>0.2 provided to hair conditioner products with improved hair conditioning performance via ease of combing. These polymers are significantly lower in aqueous viscosity as compared to cationic guar polymers with DS<0.2, such as N-Hance® cationic guar derivatives (available from Hercules Incorporated) and Jaguar® guar derivatives (available from Rhodia Corp.). Because of the higher DS and lower viscosity that the polymers of the present invention exhibit, they can be used at much higher levels in hair conditioner formulations to provide conditioning and moisturizing properties to hair. In addition, polymers of the present invention provide better salt tolerance than the counterpart guar products with low DS.

In accordance with the invention, the polymers that can be used in the invention include cationic galactomannan polymers or cationic derivatized galactomannan polymers having a weight average molecular weight (Mw) having a lower limit of 2,000 Dalton, preferably 10,000 Dalton, preferably 50,000 Dalton, more preferably 100,000 Dalton, and even more preferably 400,000 Dalton. The upper limit of the Mw of these polymers are 10,000,000 Dalton, preferably 5,000,000 Dalton, more preferably 2,000,000 Dalton, and even more preferably 1,000,000 Dalton. Examples of the polygalactomannans of this invention are guar, locust bean, honey locus, and flame tree with guar gum being the preferred source of the polygalactomannan. The preferred polygalactomannan starting material used in this invention is guar flour, guar powder, guar flakes, guar gum, or guar splits which have been derivatized with a cationic substituent.

The preferred polymers of use in this invention are cationic polygalactomannan polymers. The amount of cationic functionality on the polygalactomannan can be expressed in terms of moles of substituent. The term “degree of substitution” as used in this invention is equivalent to the molar substitution, the average number of moles of functional groups per anhydro sugar unit in the polygalactomannan gum. The cationic functionality can be present on these polymers at a DS lower limit amount of 0.25, preferably about 0.4, and more preferably 0.8. The DS upper limit is normally about 3.0, preferably about 2.0, and more preferably 1.0. In addition to molar substitution, the cationic charge on the polymers of this invention can be quantified as a charge density. The molar substitution can be converted to a charge density through a variety of methods. The preferred method for calculating charge density of cationic polymers uses a method that specifically quantifies the equivalents of quaternary ammonium groups on the polymer. Starting material having a cationic molar substitution level of 0.18 has been determined to have a charge density of 0.95 mequivalents per gram (meq/g) according to the following equation:

Cationic charge density of DS 0.18 cationic guar=(1000×0.18)/(162.14+(151.64×0.18))=0.95 meq/g.

Charge density can be measured by any method that quantifies the net positive or negative charge present on a polymer. The charge density can be determined by measurement of the moles of quaternary ammonium groups bound to the polymer backbone using standard NMR techniques of integration. This method was used for determining the charge density for polymers of use in this invention.

Cationic functionality of the polygalactomannan or derivatized polygalactomannan can be added to polygalactomannan polymer starting material by several methods. For example, the starting material can be reacted for a sufficient time and at a sufficient temperature with a tertiary amino compound or a quaternary ammonium compound containing groups capable of reacting with the reactive hydrogen ions present on the polygalactomannan or derivatized polygalactomannan in order to add the cationic functionality to the starting material. Sufficient time depends on the ingredients in the reaction mass and the temperature under which the reaction is taking place.

Cationizing agents of the present invention are defined as compounds which, by substitution reaction with hydroxy groups of the polygalactomannan can make the resultant product electrically positive. Tertiary amino compounds or various quaternary ammonium compounds containing groups capable of reacting with reactive hydrogen present on the polygalactomannan, can be used, such as 2-dialkylaminoethyl chloride and quaternary ammonium compounds such as 3-chloro-2-hydroxypropyltrimethylammonium chloride, and 2,3-epoxy-propyltrimethylammonium chloride. Preferred examples include glycidyltrialkylammonium salts and 3-halo-2-hydroxypropyltrialkylammonium salts such as glycidyltrimethylammonium chloride, glycidyltriethylammonium chloride, gylcidyltripropylammonium chloride, glycidylethyldimethylammonium chloride, glycidyidiethylmethylammonium chloride, and their corresponding bromides and iodides; 3-chloro-2-hydroxypropyltrimethylammonium chloride, 3-chloro-2-hydroxypropyltriethylammonium chloride, 3-chloro-2-hydroxypropyltripropylammonium chloride, 3-chloro-2-hydroxypropylethyldimethylammonium chloride, and their corresponding bromides and iodides; and quaternary ammonium compounds such as halides of imidazoline ring containing compounds.

Other derivatization of cationic polygalactomannan with nonionic substituents, i.e., hydroxyalkyl wherein the alkyl represents a straight or branched hydrocarbon moiety having 1 to 6 carbon atoms (e.g., hydroxyethyl, hydroxypropyl, hydroxybutyl) or anionic substituents, such as carboxymethyl groups are optional. These optional substituents are linked to the polygalactomannan molecule by reaction of the polygalactomannan molecule with reagents such as (1) alkylene oxides (e.g., ethylene oxide, propylene oxide, butylene oxide) to obtain hydroxyethyl groups, hydroxypropyl groups, or hydroxybutyl groups, or with (2) chloromethyl acetic acid to obtain a carboxymethyl group on the polygalactomannan molecule. This reaction can take place when the polygalactomannan is in the “split”, “flour” or any other physical form. The process for preparing derivatized polygalactomannan is well known in the art.

Hair conditioner products includes hair conditioners, leave-on hair conditioner, leave-in conditioner, rejuvenating conditioner, crème rinse, oil-free hair conditioners, rinse-off hair conditioner, conditioning rinse, foaming conditioner, conditioning styling gel, conditioning mousse, spay-on conditioner, hair dressing crème and hair repair spray.

Conditioning agents include any material which is used to give a particular conditioning benefit to hair. Suitable conditioning agents are those which deliver one or more benefits relating to shine, softness, comb ability, antistatic properties, wet-handling, damage, manageability, body, and greasiness. The conditioning agents typically comprise a water insoluble, water dispersible, non-volatile, liquid that forms emulsified, liquid particles. Suitable conditioning agents are characterized generally as silicones (e.g. silicone oils, cationic silicones, silicone gums, high refractive silicones, and silicone resins), organic conditioning oils (e.g. hydrocarbon oils, polyolefins, fatty acids, fatty alcohols, and fatty esters) or combinations thereof. Such conditioning agents should be physically and chemically compatible with the essential components of the composition, and should not otherwise unduly impair product stability, aesthetics or performance.

The concentration of the conditioning agent in the hair conditioner products should be sufficient to provide the desired conditioning benefits, and as will be apparent to one of ordinary skill in the art. Such concentration can vary with the conditioning agent, the conditioning performance desired, the average size of the conditioning agent particles, the type and concentration of other components, and other like factors.

Examples of active agents that may suitably be included in the hair conditioner products according to the present invention are the following.

1) Perfumes, which give rise to an olfactory response in the form of a fragrance and deodorant perfumes which in addition to providing a fragrance response can also reduce malodor.

2) Emollients, such as isopropylmyristate, silicone materials, mineral oils, vegetable oils and fats, animal oils and fats which give rise to a tactile response in the form of an increase in hair lubricity.

3) Moisturizing agents, that keep the hair moist by either adding moisture or preventing from evaporating from the hair.

4) Hair soothing agents, including panthenoic acid derivatives, (e.g., panthenol, dexpanthenol and ethyl panthenol), aloe vera, pantothenic acid and its derivatives, allantoin, bisabolol, and dipotassium glycyrrhizinate), retinoids, (e.g. retinol palmitate), tocopheryl nicotinate, skin treating agents, vitamins and derivatives thereof. These hair soothing agents are preferably used in concentrations from about 0.1% to about 30%, more preferably from about 0.5% to about 20%, still more preferably from about 0.5% to about 10%, by weight of the composition.

5) Anti-Oxidants/Radical Scavengers which are used in hair conditioner products at concentrations of which range from about 0.1% to about 10%, more preferably from about 1% to about 5%, by weight of the composition. Examples of anti-oxidants or radical scavengers for use herein include ascorbic acid and its salts, ascorbyl esters of fatty acids, ascorbic acid derivatives (e.g., magnesium ascorbyl phosphate, sodium ascorbyl phosphate, ascorbyl sorbate), tocopherol, tocopherol acetate, other esters of tocopherol, butylated hydroxy benzoic acids and their salts, 6-hydroxy-2,5,7,8-tetramethylchroman-2-carboxylic acid (commercially available under the trademark Trolox® antioxidant, from Oxis International Inc.), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts may be used.

6) Chelators or chelating agents, refers to those hair benefit agents which are capable of removing a metal ion from a system by forming a complex so that the metal ion cannot readily participate in or catalyze chemical reactions. The chelating agents as hair benefit agents for use herein are preferably formulated at concentrations ranging from about 0.1% to about 10%, more preferably from about 1% to about 5%, by weight of the composition. Preferred chelating agents for use in the active phase of the compositions of the present invention include furildioxime, furilmonoxime, and derivatives thereof.

7) Anti-Inflammatory agents are used in hair conditioner products at concentrations in the range from about 0.1% to about 10%, more preferably from about 0.5% to about 5%, by weight of the composition. The anti-inflammatory agents may comprise steroids. Examples of steroidal anti-inflammatory agents suitable for use herein include corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use is hydrocortisone.

Nonsteroidal anti-inflammatory agents are also suitable for use herein as skin benefit agents in the active phase of the compositions.

8) Antimicrobial actives are used in hair conditioner products at concentrations in the range of from about 0.001% to about 10%, more preferably from about 0.01% to about 5%, and still more preferably from about 0.05% to about 2%, by weight of the compositions. Examples of antimicrobial actives for use herein includes .beta.-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione, clotrimazole, and combinations thereof.

9) Sunscreen actives of use in hair conditioning products may be either organic or inorganic in nature. Among the inorganic sunscreens useful hererin are metallic oxides such as titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof.

Examples of organic sunscreen actives include p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane. Among these sunscreens, preferred are 2-ethylhexyl-p-methoxycinnamate (commercially available as PARSOL® MCX sunscreen from Givaudan Roure corporation), 4,4′-t-butyl methoxydibenzoyl-methane (commercially available as PARSOL® 1789 sunscreen from Givaudan Roure corporation), 2-hydroxy-4-methoxybenzophenone, octyidimethyl-p-aminobenzoic acid, digalloyltrioleate, 2,2-dihydroxy-4-methoxybenzophenone, ethyl-4-(bis(hydroxypropyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene and combinations thereof.

10) Antidandruff agent which removes dandruff from hair and scalp.

11) Styling agent which provide hair styling.

12) Hair bodying and volumizing agents which increases hair body and volume.

The above list of hair conditioner active ingredients is provided as examples and not a complete list of active ingredients that can be used. Other ingredients that are used in these types of products are well known in the industry. In addition to the above ingredients conventionally used, the composition according to the present invention can optionally also include ingredients such as a colorant, preservative, nutritional supplements, activity enhancer, emulsifiers, functional polymers, viscosifying agents (such as NaCl, NH₄Cl, KCl, Na₂SO₄, fatty alcohols, fatty acid esters, fatty acid amides, fatty alcohol polyethyleneglycol ethers, sorbitol polyethyleneglycol ethers, cocamide monoethanolamide, cocamide diethanolamide, cocamidopropyl betaine, clays, silicas, cellulosic polymers, and xanthan), suspending agents (such as clays, silica, and xanthan), stabilizers, alcohols having 1-6 carbons, fats or fatty compounds, zinc pyrithione, silicone material, hydrocarbon polymer, oils, medicaments, flavors, fragrances, rejuvenating reagents, and mixtures thereof.

In accordance with the present invention, examples of functional polymers that can be used in blends with the cationic polygalactomannan or derivatives thereof of this invention include water-soluble polymers such as anionic, hydrophobically-modified, and amphoteric acrylic acid copolymers, vinylpyrrolidone homopolymers; cationic, hydrophobically-modified, and Company), Structure® Plus (Acrylates/Aminoacrylates/C10-30 Alkyl Peg 20 Itaconate copolymer, National Starch and Chemical Company), Quatrisoft® LM-200 polymer (Polyquaternium-24, available from Amerchol Corporation.), the metal oxides of titanium, zinc, iron, zirconium, silicon, manganese, aluminium and cerium, polycarbonates, polyethers, polyethylenes, polypropylenes, polyvinyl chloride, polystyrene, polyamides, polyacrylates, cyclodextrins and mixtures thereof.

Cyclodextrins are solubilized, water-soluble, uncomplexed cyclodextrins. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. Examples of preferred water-soluble cyclodextrin derivatives suitable for use herein are hydroxypropyl alpha-cyclodextrin, methylated alpha-cyclodextrin, methylated beta-cyclodextrin, hydroxyethyl beta-cyclodextrin, and hydroxypropyl beta-cyclodextrin. Cyclodextrins particularly preferred for use herein are alpha cyclodextrin, beta cyclodextrin, hydroxypropyl alpha cyclodextrin, hydroxypropyl beta cyclodextrin, and mixtures thereof.

The hair conditioner compositions are “substantially surfactant-free” and provide little or no detersive or cleaning effect on hair. Surfactants which do provide detersive effects are known in the art and include anionic, nonionic, cationic, zwitterionic, amphoteric or mixtures thereof. The substantially surfactant-free hair conditioner compositions contain less than 1% by weight of a surfactant which is to provide cleaning performance to a composition. Preferably the “substantially surfactant-free” hair conditioner compositions contains less than 0.5% by weight of a surfactant, more preferably less than 0.1% by weight of a surfactant.

For a more detailed understanding of the invention, reference can be made to the following examples which are intended as further illustration of the invention and are not to be construed in a limiting sense. All parts and percentages are by weight unless stated otherwise.

### EXAMPLE 1

Hair Conditioner Formulation: The polymers of investigation were formulated into the following formulation to evaluate for their wet and dry combing performance.

| | |
|---|---|
| 90.94 g | Deionized water |
| 00.70 g | Natrosol ® 250HHR hydroxyethylcellulose, available from |
| | Hercules Incorporated |
| 00.20 g | Polymer of this invention or commercial polymer |
| 02.00 g | Cetyl alcohol |
| 00.50 g | Potassium Chloride |
| 02.00 g | Isopropyl Myristate |
| As required | citric acid to adjust pH |
| As required | Sodium hydroxide to adjust pH |
| 00.50 g | Preservative |

The conditioner formulation was prepared by first adding Natrosol® hydroxyethyl cellulose type 250 HHR to water in a beaker and under agitation to form a slurry. Next, pH of the slurry was adjusted to 8.0 to 8.5. The slurry was stirred for about 30 minutes or until the polymer fully dissolved. Next, the polymer of this invention or a commercial polymer listed in Table 1 was added to the beaker and mixed for 30 more minutes. The solution was heated to about 65° C. and stirred until it became smooth. Next, cetyl alcohol was added to the beaker and mixed until it appeared homogeneously mixed. The mixture was cooled to about 50° C. while mixing and then potassium chloride was added. Next, isopropyl myristate was added and mixed until the mixture looked homogeneous. The pH of the mixture was adjusted between 5.25 and 5.5 with citric acid and/or NaOH solution. The conditioner was preserved with 0.5% preservative and mixing was continued until it reached room temperature. The conditioner was tested for its combing performance. All conditioners including the controls with commercial polymers contained 0.7% Natrosol® hydroxyethyl cellulose type 250 HHR. All polymers under evaluations were used at 0.2% level.

To test the conditioner for combing, flat tresses of mildly bleached European hair weighing about 3 grams were used in the study. The hair tresses were obtained from International Hair Importers and Products Inc. of Glendale, N.Y. The hair tresses were first cleaned with 4.5% active sodium lauryl sulfate solution. To clean the hair tresses, the hair tresses were first wetted with 40° C. tap water and then 5.0 ml of sodium lauryl sulfate solution was applied along each of the tresses length. Each tress was kneaded for 30 seconds. Each tress was then rinsed under 40° C. running water for 30 seconds. The hair cleaning step was repeated followed by rinsing with room temperature tap water for 30 seconds. Each tress was then dried overnight.

Next day, the sample tress was rewetted with 40° C. tap water and then 0.5 gram conditioner per gram of hair was applied uniformly along the length of hair. The tress was kneaded for 30 seconds and then it was rinsed under 40° C. running water for 30 seconds. Same amount of conditioner was reapplied again along the length of the tress and the tress was kneaded again for 30 seconds, and then it was rinsed under 40° C. running water for 30 seconds. The tress was rinsed with room temperature tap water for 30 seconds. The tress was combed immediately for eight times on an Instron instrument to measure wet combing energy (gf-mm/g). During the wet combing, the hair was sprayed with water immediately before each combing cycle to keep it wet. From the 8 combing cycle data, average wet combing energy in gram force-mm/gram of hair (gf-mm/g) was calculated. The tress was then stored overnight at about 50% relative humidity and about 23° C. For combing the tresses, an ACE rubber comb, style 61636, from Goody Products, Inc., Atlanta, Ga., was used.

Next day, the tress was first combed with fine teeth rubber comb to free-up hair stuck together. Again, the hair tress was combed eight times on an Instron instrument to determine average force required to comb one gram of dry hair. The higher the value on the Instron, the poorer the conditioning effect of the polymer being tested. Two tresses were used per hair conditioning formulation. The data reported in Table 1 are based on an average of two tresses.

TABLE 1

| | % | % | | | | |
|---|---|---|---|---|---|---|
| | Polymer | Commercial | | Conditioner | Wet | Dry |
| | of This | Cationic | Polymer | Viscosity | Combing | Combing |
| EX # | Invention | Polymer | Level | (cps) | (gf-mm/g) | (gf-mm/g) |
| Ex#1A | 0.0 | 0.0 | 0 | 990 | 4774 | 287 |
| Ex#1B | 0.0 | 0.0 | 0 | 1380 | 4513 | 364 |
| Ex#1C | | N-Hance ® 3269 | 0.2 | 1330 | 1389 | 263 |
| Ex#1D | | AguaCat ® CG518 | 0.2 | 1100 | 1142 | 332 |
| Ex#1E | EX 1 | 0.0 | 0.2 | 1930 | 889 | 124 |
| EX#1F | EX 2 | 0.0 | 0.2 | 1700 | 530 | 140 |
| Ex#1G | | UCARE ® LR400 | 0.2 | 1280 | 811 | 1116 |
| EX#1H | | UCARE ® JR30M | 0.2 | 1960 | 868 | 849 |

Ingredients used in the tests and listed in Table 1:
(1) Natrosol ® 250HHR: Hydroxyethyl cellulose from Hercules Incorporated Wilmington, DE
(2) UCARE ® LR400 HEC: Cationic HEC from Dow Chemicals, Midland, MI
(3) UCARE ® JR30M HEC: Cationic HEC from Dow Chemicals, Midland, MI
(4) N-Hance ® 3269 cationic guar, cationic DS 0.13, Weight average Molecular weight 500,000 from Hercules Incorporated, Wilmington, DE
(5) AquaCat ® CG 518: cationic guar, cationic DS 0.18, Weight average Molecular weight 50,000 from Hercules Incorporated, Wilmington, DE
(6) EX 1: Polymer of this invention, cationic guar, cationic DS 0.51: Weight average Molecular weight 1.2 MM from Hercules Incorporated. Wilmington, DE
(7) EX 2: Polymer of this invention, cationic guar cationic DS 1.06.
(8) Crodacol ™ C95NF: Cetyl alcohol from Croda Inc. Parsippany, NJ
(9) KCI: Potassium chloride
(10) Stepan ® IPM: Isopropyl myristate from Stepan Company, Northfield, IL
(11) Germaben ® II: preservative from ISP Wayne, NJ

Example 1A and 1B in Table 1 provide wet and dry combing data for the hair conditioning formulation without any type of cationic polysaccharide. The energy (gf-mm/g) required to comb wet hair is about 4500-4800. With the addition of 0.2% commercial cationic guar, Example #1C and Example #1D, the wet combing energy was about 1100-1400 gf-mm/g of hair and the dry combing energy was about the same as with the no cationic polysaccharide of Examples 1A and 1B. With the commercial cationic hydroxyethyl cellulose of Examples 1G and 1H, the wet combing energy was about 800-900 gf-mm/g of hair. However, the dry combing energy was much higher, 850-1100 gf-mm/g of hair, higher than even the conditioner without the conditioning polymer mentioned in Example 1A and 1B. The polymer of this invention not only reduced the wet combing energy to the range of 500 to 900 gf-mm/g, but also it reduced the dry combing energy to about 125-150 gf-mm/g.

### EXAMPLE 2

Hair Conditioner Formulation: The cationic guar polymers of this invention designated as EX 1, EX 2, as previously described in Table 1 and EX 3 were formulated into the following conditioner formulation to evaluate for their performance during the hair lathering, hair rinsing, wet combing and dry combing against the commercially available cationic guar

| | |
|---|---|
| 94.35 g | Deionized water |
| 01.25 g | Hydroxyethyl Cellulose (Natrosol ® 250HHR HEC |
| | available from Hercules Incorporated) |
| 00.20 g | Polymer of this invention or commercial polymer |
| 04.10 g | Stearalkonium chloride, Ammonyx ® 4002 Stepan Co. |
| 00.10 g | Preservative, methylcholoroisothiazolinone and |
| | methylisothiazolinone; Kathon ™ preservative, |
| | available from Rhom & Haas Co. |
| As required | citric acid to adjust pH |

The conditioner formulation was prepared by first adding Natrosol® hydroxyethyl cellulose type 250 HHR to water in a beaker and under agitation to form slurry. Next, pH of the slurry was adjusted to 8.0 to 8.5. The slurry was stirred for about 30 minutes or until the polymer fully dissolved. Next, the polymer of this invention or a commercial polymer listed in Table 2 was added to the beaker containing Natrosol solution and then pH was lowered to about 6 with citric acid, and the polymer solution was mixed for 30 more minutes. The solution was heated to about 50° C. in a water-bath and stirred at 50° C. for about 45 minutes. Next stearalkonium chloride (Ammonyx® LO, available from Stepan Co.) was added to the solution and allowed to mix for 30 minutes. The solution was removed from the 50° C. water-bath and cooled to room temperature while stirring. The solution pH was adjusted to about 4 to 6 with citric acid. Next, preservative was added and mixed for 15 minutes. The conditioner solution was reweighed and adjusted for ant water-loss.

Unlike the quantitative testing performed on the conditioner samples in Example 1, the conditioners were subjectively tested for their (a) Wet-Feel during the lathering/application to wet hair (b) Wet-Feel during the rinsing cycle of the hair (c) Easy of combing of wet hair (d) dry combing of hair dried overnight. Subjective ranking used was as follows. A higher the ranking, the better the product performance.

1 = No conditioning, squeaky wet feel
2 = Light conditioning
3 = Conditioning
4 = Heavy conditioning

To test the conditioner for combing, flat tresses of mildly bleached European hair weighing about 3 grams were used in the study. Two tresses were used for each formulation. The hair tresses were obtained from International Hair Importers and Products Inc. of Glendale, N.Y.

Preparation of Hair for conditioner Treatment: The hair tresses were first cleaned with 4.5% active sodium lauryl sulfate solution. To clean the hair tresses, the hair tresses were first wetted with 40° C. tap water and then 5.0 ml of sodium lauryl sulfate solution was applied along each of the tresses length. Each tress was kneaded for 30 seconds. Each tress was then rinsed under 40° C. running water for 30 seconds. The hair cleaning step was repeated followed by rinsing with room temperature tap water for 30. Each tress was then dried overnight.

Treatment with Conditioner: Next day the tress was wetted by holding vertically under running 40° C. tap water. Next, the tress was placed into a plastic weighing dish, curling in a circular manner. Then, 0.50 ml of the conditioner solution_for each gram of hair tress was applied evenly along the length of the tress with 5 ml syringe. The conditioner solution was further distributed evenly in the tress gently with the fingertips. The application took about 15 seconds. The tress was removed from the dish and held vertically. The tress was kneaded thoroughly for 30 seconds, alternately squeezing gently between the fingertips along the length of the tress, and stroking the tress to distribute the solution uniformly. The hair wet feel was noted at this lathering time for the (a) subjective evaluation. A higher the ranking better the wet feel during the lathering.

The tress was than rinsed under 40° C. running tap water while kneading as above for 30 seconds. The wet feel during rinse-off was noted at this time for the (b) subjective evaluation. A higher the ranking better the wet feel during rinse-off. Hairs were rinsed with running room temperature Deionized water for 30 seconds. After the washing with Deionized water, the excess water was squeezed out twice with the fingers.

Next hair was combed using an Ace® brand #62746 comb (coarse and fine teeth). The hair tress was combed 3 times with the coarse end of the comb, followed by 3 times with the fine end. The level of ease of combing was noted for subjective evaluation. A higher the ranking, easier the wet combing. The hair tresses were hung overnight to dry in the CT room (73° F., 50% RH). Using the fine end of an Ace® brand #62746 comb, hair tress were combed 3 times. The level of ease of combing was noted for subjective evaluation. A higher the ranking, easier the dry combing.

TABLE 2

| | % Polymer | % Commercial | Polymer | Polymer Mol | | Hair Wet Feel | | | |
|---|---|---|---|---|---|---|---|---|---|
| | of This | Cationic | Cationic DS | Wt (approx.) | Polymer | during | Hair Wet Feel | Wet | Dry |
| EX # | Invention | Polymer | (appx.) | MM | Level | lathering | during Rinsing | Combing | Combing |
| Ex#2A | — | — | 0.0 | — | 0 | 2 | 1 | 1 | 2 |
| Ex#2B | — | N-Hance ® 3215 | 0.2 | 1.2 | 0.2 | 3 | 2 | 2 | 3 |
| Ex#2C | EX 1 | — | 0.5 | 1.1 | 0.2 | 3 | 3 | 3 | 3 |
| Ex#2D | EX 2 | — | 1.1 | 1.7 | 0.2 | 3 | 3 | 3 | 4 |
| Ex#2E | EX 3 | — | 1.8 | 1.9 | 0.2 | 3.5 | 3.5 | 3.5 | 4 |

In the subjective combing test, cationic guar with higher degree of cationic substitution polymer of this invention overall provided better hair wet feel during lathering, better wet feel during rinsing cycle, easier wet combing and easier dry combing than the conditioner with commercial N-Hance® cationic guar, available from Hercules Incorporated.

### EXAMPLE 3

Hair Conditioner Formulation: The cationic guar polymers of this invention designated as EX 1, EX 2 and EX 3 were formulated into the following conditioner formulation to evaluate for their performance during the hair lathering, hair rinsing, wet combing and dry combing against the commercially available cationic guar

| | |
|---|---|
| 94.50 g | Deionized water |
| 04.50 g | Cetearyl alcohol and ceteareth-20, Promulgen ® D from |
| | Noveon Corp |
| 00.50 g | Glyceryl stearate, Emerest ® 2400 from Cognis Corp. |
| 00.20 g | PEG 5M Polyox ® WSR N-80 from Dow Chemicals |
| 00.20 g | Polymer of this invention or a commercial cationic guar |
| 00.10 g | Preservative, methylcholoroisothiazolinone and |
| | methylisothiazolinone; Kathon ® Rhom & Haas Co. |
| As required | citric acid to adjust pH |

The conditioner formulation was prepared by first adding the polymer of this invention or a commercial polymer listed in Table 3 was added to the vortex of well agitated water in a beaker. Next, pH was lowered to about 6 with citric acid, and the polymer solution was mixed for 30 more minutes. The solution was heated to about 75° C. in a water-bath and stirred at 75° C. This was a water-phase. In a separate beaker, oil phase prepared by melting glyceryl stearate, PEG and cetearyl alcohol and ceteareth-20 at 75° C. Next, the oil phase was slowly added to the water phase under agitation at 75° C. Mixing was continued for 10 minutes. The emulsion was removed from the water-bath and cooled to room temperature while mixing. The emulsion pH was adjusted to about 5 with citric acid. Next, the preservative was added and mixed for 15 minutes. The conditioner solution was reweighed and adjusted for water-loss.

The conditioners were subjectively tested for their (a) Wet-Feel during the lathering/application to wet hair (b) Wet-Feel during the rinsing cycle of the hair (c) Easy of combing of wet hair (d) dry combing of hair dried overnight. Subjective ranking used was as follows. A higher the ranking, the better the product performance.

1 = No conditioning, squeaky wet feel
2 = Light conditioning
3 = Conditioning
4 = Heavy conditioning

To test the conditioner for combing, flat tresses of mildly bleached European hair weighing about 3 grams were used in the study. Two tresses were used for each formulation. The hair tresses were obtained from International Hair Importers and Products Inc. of Glendale, N.Y. The hair tress evaluation was conducted using the method described in Example 2.

TABLE 3

| | % Polymer | % Commercial | Polymer | Polymer Mol | | Hair Wet Feel | | | |
|---|---|---|---|---|---|---|---|---|---|
| | of This | Cationic | Cationic DS | Wt (approx.) | Polymer | during | Hair Wet Feel | Wet | Dry |
| EX # | Invention | Polymer | (appx.) | MM | Level | lathering | during Rinsing | Combing | Combing |
| Ex#3A | — | — | 0.0 | — | 0 | 1 | 1 | 1 | 1 |
| Ex#3B | — | N-Hance ® 3215 | 0.2 | 1.2 | 0.2 | 3 | 3 | 3 | 4 |
| Ex#3C | EX 1 | — | 0.5 | 1.1 | 0.2 | 3 | 3 | 3 | 4 |
| Ex#3D | EX 2 | — | 1.1 | 1.7 | 0.2 | 3 | 3 | 3 | 4 |
| Ex#3E | EX 3 | — | 1.8 | 1.9 | 0.2 | 3. | 3 | 3. | 4 |

In our subjective combing test, cationic guar based formulation prepared with higher cationic degree of substitution polymer of this invention or with the commercial cationic guar performed equal but better than a conditioner with no cationic guar polymer.

### EXAMPLE 4

Hair Conditioner Formulation: The cationic guar polymers of this invention designated as EX 1, EX 2 and EX 3 were formulated into the following conditioner formulation to evaluate for their performance during the hair lathering, hair rinsing, wet combing and dry combing against the commercially available cationic guar.

| | |
|---|---|
| 94.35 g | Deionized water |
| 01.50 g | Hydroxyethylcellulose, Natrosol ® 250HHR from |
| | Hercules, Inc. |
| 00.20 g | Tetrasodium EDTA, Versene ® 220 from Dow Chemicals. |
| 00.30 g | Cetrimonium chloride Ammonyx CETAC from Stepan Co. |
| 01.00 g | Cetearyl alcohol, Crodacol ® CS50, from Croda |
| 01.00 g | Glyceryl stearate and PET 100 stearate, Arlacel ® from |
| | Uniqema. |
| 02.00 g | Silicone emulsion, Dow Corning 1784 from Dow Corning |
| 00.60 g | Polymer of this invention or a commercial cationic guar |
| 00.40 g | Preservative, DM DM Hydantoin, Glydant from Lonza. |
| As required | citric acid to adjust pH |

The conditioner formulation was prepared by first adding Natrosol® hydroxyethyl cellulose type 250 HHR to water in a beaker and under agitation to form slurry. Next, pH of the slurry was adjusted to 8.0 to 8.5. The slurry was stirred for about 30 minutes or until the polymer fully dissolved. Next, the polymer of this invention or a commercial polymer listed in Table 4 was added to the beaker containing Natrosol solution and then pH was lowered to about 6 with citric acid, and the polymer solution was mixed for 30 more minutes. The solution was heated to about 75° C. in a water-bath and stirred at 75° C. Next, tetrasodium EDTA, cetrimonium chloride, cetearyl alcohol, glyceryl stearate and PET 100 stearate were added. Between each addition, the formulation was mixed for 10 minutes. The solution was removed from the 75° C. water-bath and cooled to room temperature while stirring. Next, Silicone emulsion was added and followed by preservative. The solution pH was adjusted to about 5 with citric acid. The conditioner solution was reweighed and adjusted for any water-loss.

The conditioners were subjectively tested for their (a) Wet-Feel during the lathering/application to wet hair (b) Wet-Feel during the rinsing cycle of the hair (c) Easy of combing of wet hair (d) dry combing of hair dried overnight. Subjective ranking used was as follows. A higher the ranking, the better the product performance.

1 = No conditioning, squeaky wet feel
2 = Light conditioning
3 = Conditioning
4 = Heavy conditioning

To test the conditioner for combing, flat tresses of mildly bleached European hair weighing about 3 grams were used in the study. Two tresses were used for each formulation. The hair tresses were obtained from International Hair Importers and Products Inc. of Glendale, N.Y. The hair tress evaluation was conducted using the method described in example 2.

TABLE 4

| | | | | Polymer | | | | | |
|---|---|---|---|---|---|---|---|---|---|
| | Polymer | | Polymer | Mol Wt | | Hair Wet | Hair Wet | | |
| | of This | Comm. Cationic | Cationic DS | MM | Polymer | Feel | Feel | Wet | Dry |
| EX # | Invention | Polymer | approx | (aprox) | Level | during lathering | during Rinsing | Combing | Combing |
| Ex#4A | — | — | 0.0 | — | 0 | 2 | 2 | 2 | 3 |
| Ex#4B | — | N-Hance ® 3215 | 0.2 | 1.2 | 0.6 | 2 | 3 | 3 | 4 |
| Ex#4C | EX 1 | — | 0.5 | 1.1 | 0.6 | 3 | 3 | 3 | 4 |
| Ex#4D | EX 2 | — | 1.1 | 1.7 | 0.6 | 3 | 3 | 3 | 4 |
| Ex#4E | EX 3 | — | 1.8 | 1.9 | 0.6 | 3. | 3 | 3. | 4 |

In subjective combing test, cationic guar based formulation prepared with higher cationic degree of substitution polymer of this invention a very slight improvement hair wet feel during the lathering was observed over the commercial cationic guar.

Although the invention has been described with referenced to preferred embodiments, it is to be understood that variations and modifications in form and detail thereof may be made without departing from the spirit and scope of the claimed invention. Such variations and modifications are to be considered within the purview and scope of the claims appended hereto.

amphoteric vinylpyrrolidone copolymers; nonionic, cationic, anionic, and amphoteric cellulosic polymers such as hydroxyethylcellulose, hydroxypropylcellulose, carboxymethylcellulose, hydroxypropylmethylcellulose, cationic hydroxyethylcellulose, cationic carboxymethylhydroxyethylcellulose, and cationic hydroxypropylcellulose; acrylamide homopolymers and cationic, amphoteric, and hydrophobically-modified acrylamide copolymers, polyethylene glycol polymers and copolymers, hydrophobically-modified polyethers, hydrophobically-modified polyetheracetals, hydrophobically-modified polyols and polyetherurethanes and other polymers referred to as associative polymers, hydrophobically-modified cellulosic polymers, polyethyleneoxide—propylene oxide copolymers, and nonionic, anionic, hydrophobically-modified, amphoteric, and cationic polysaccharides such as xanthan, chitosan, carboxymethyl guar, alginates, hydroxypropyl guar, carboxymethyl guar hydroxypropyltrimethylammonium chloride, guar hydroxypropyltrimethylammonium chloride, hydroxypropyl guar hydroxypropyltrimethylammonium chloride.

In accordance with the invention, the silicone materials which can be used are, in particular, polyorganosiloxanes that are insoluble in the composition and can be in the form of polymers, oligomers, oils, waxes, resins, or gums.

The organopolysiloxanes are defined in greater detail in Walter Noll's “Chemistry and Technology of Silicones” (1968) Academic Press. They can be volatile or non volatile.

If volatile, the silicones are more particularly chosen from those having a boiling point in the range of between about 60° C. to about 260° C.

If the silicone is a cyclic silicone, the cyclic silicones of use may comprise from 3 to 7 and preferably from 4 to 5 silicon atoms. These are, for example, octamethylcyclotetrasiloxane were sold in particular under the name “Volatile Silicone 7207” by Union Carbide or “Silbione 70045 V 2” by Rhone Poulenc, decamethyl cyclopentasiloxane were sold under the name “Volatile Silicone 7158” by Union Carbide, and “Silbione 70045 V 5” by Rhone Poulenc, and mixtures thereof.

Mention may also be made of mixtures of cyclic silicones with organosilicone compounds, such as the mixture of octamethylcyclotetrasiloxane and tetratrimethylsilylpentaerythritol (50/50) and the mixture of octamethylcyclotetrasiloxane and oxy I,I′ bis(2,2,2′,2′,3,3′ hexatrimethylsilyloxy) neopentane.

If the silicone is a linear silicone, the linear volatile silicone may comprise from 2 to 9 silicon atoms and having a viscosity of less than or equal to 5×10⁻⁶ m²/s at 25° C. An example is decamethyltetrasiloxane was sold in particular under the name “SH 200” by Toray Silicone company. Silicones belonging to this category are also described in the article published in Cosmetics and Toiletries, Vol. 91, January 76, pp. 27 32, Todd & Byers “Volatile Silicone Fluids for Cosmetics”.

Non volatile silicones, and more particularly polyarylsiloxanes, polyalkylsiloxanes, polyalkylarylsiloxanes, silicone gums and resins, polyorganosiloxanes modified with organofunctional groups, and mixtures thereof, are preferably used.

In accordance with the invention, the silicone polymers and resins which can be used are, in particular, polydiorganosiloxanes having high number-average molecular weights of between 200,000 and 1,000,000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Examples of these silicone polymers and resins are as follows:

Polydimethylsiloxane,

polydimethylsiloxanes/methylvinylsiloxane gums,

polydimethylsiloxane/diphenylmethylsiloxane,

polydimethylsiloxane/phenylmethylsiloxane, and

polydimethylsiloxane/diphenylsiloxanemethylvinylsiloxane.

Silicone products which can be used more particularly in accordance with the invention are mixtures such as:

(a) mixtures formed from a polydimethylsiloxane hydroxylated at the end of the chain (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the Dow Corning Company;

(b) mixtures formed from a polydimethylsiloxane gum with a cyclic silicone, such as the product SF 1214 Silicone Fluid from the company General Electric Company; this product is an SF 30 gum corresponding to a dimethicone, having a number average molecular weight of 500,000, dissolved in SF 1202 Silicone Fluid oil corresponding to decamethylcyclopentasiloxane; and

(c) mixtures formed of two PDMSs of different viscosities, and more particularly of a PDMS gum and a PDMS oil, such as the product SF 1236 from the General Electric Company. The product SF 1236 is a mixture of a gum SE 30 defined above, having a viscosity of 20 m2/s, and an oil SF 96, with a viscosity of 5×10-6 m2/s. This product preferably contains 15% SE 30 gum and 85% SF 96 oil.

These silicone materials function as conditioning agents for skin surfaces. Other types of conditioning agents include oils, waxes, hydrocarbon oils, such as mineral oil and fatty acid ester of glycerol, and panthenol and its derivatives, such as panthenyl ethyl ether, panthenyl hydroxypropyl steardimonium chloride, and pantothenic acid.

Oils include hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin and its derivatives, wax esters, beeswax derivatives, sterols and phospholipids, and combinations thereof.

Examples of hydrocarbon oils and waxes suitable for use herein include petrolatum, mineral oil, micro-crystalline waxes, polyalkenes, paraffins, cerasin, ozokerite, polyethylene, perhydrosqualene, poly alpha olefins, hydrogenated polyisobutenes and combinations thereof.

Examples of silicone oils suitable for use herein include dimethicone copolyol, dimethylpolysiloxane, diethylpolysiloxane, mixed C₁-C₃₀ alkyl polysiloxanes, phenyl dimethicone, dimethiconol, and combinations thereof. Preferred are non-volatile silicones selected from dimethicone, dimethiconol, mixed C₁-C₃₀ alkyl polysiloxane, and combinations thereof. Non-limiting examples of silicone oils useful herein are described in U.S. Pat. No. 5,011,681, incorporated herein by reference in its entirety.

Examples of diglycerides and triglycerides suitable for use herein include castor oil, soy bean oil, derivatized soybean oils, safflower oil, cotton seed oil, corn oil, walnut oil, peanut oil, olive oil, cod liver oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, and combinations thereof. In addition any of the above oils that have been partially or fully hydrogenated are also suitable.

Examples of acetoglyceride esters suitable for use herein include acetylated monoglycerides.

Examples of alkyl esters suitable for use herein include isopropyl esters of fatty acids and long chain esters of long chain fatty acids, e.g. SEFA (sucrose esters of fatty acids). Lauryl pyrolidone carboxylic acid, pentaerthritol esters, aromatic mono, di or triesters, cetyl ricinoleate, non-limiting examples of which include isopropyl palmitate, isopropyl myristate, cetyl riconoleate and stearyl riconoleate. Other examples are: hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Examples of alkenyl esters suitable for use herein include oleyl myristate, oleyl stearate, oleyl oleate, and combinations thereof.

Examples of lanolin and lanolin derivatives suitable for use herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, hydroxylated lanolin, hydrogenated lanolin and combinations thereof.

Still other suitable oils include milk triglycerides (e.g., hydroxylated milk glyceride) and polyol fatty acid polyesters.

Still other suitable oils include wax esters, non-limiting examples of which include beeswax and beeswax derivatives, spermaceti, myristyl myristate, stearyl stearate, and combinations thereof. Also useful are vegetable waxes such as carnauba and candelilla waxes; sterols such as cholesterol, cholesterol fatty acid esters; and phospholipids such as lecithin and derivatives, sphingo lipids, ceramides, glycosphingo lipids, and combinations thereof.

The suitable stabilizers include Pemulen® TR-1 polymer (Acrylates/C10-30 Alkyl Acrylate Crosspolymer, available from Noveon, Inc.), Pemulen® TR-2 polymer (Acrylates/C10-30 Alkyl Acrylate Crosspolymer, available from Noveon, Inc.), Carbolpol® ETD 2020 polymer (Acrylates/C10-30 Alkyl Acrylate Crosspolymer, available from Noveon, Inc.), Carbopol® 1382 polymer (Acrylates/C10-30 Alkyl Acrylate Crosspolymer, available from Noveon, Inc.), Natrosol® CS Plus 330 hydroxyethylcellulose, 430, Polysurf® 67Cetyl Hydroxyethyl Cellulose, available from Hercules Incorporated), Aculyn™ 22 rheology modifier (Acrylates/Steareth-20 Methacrylate Copolymer, available from Rohm&Haas Company) Aculyn™ 25 rheology modifier (Acrylates/Laureth-25 Methacrylate copolymer, available from Rohm&Haas Company), Aculyn™ 28 rheology modifier (Acrylates/Beheneth-25 Methacrylate copolymer, available from Rohm&Haas Company), Aculyn™ 46 rheology modifier (Peg-150/Stearyl Alcohol/SMDI copolymer, available from Rohm&Haas Company) Stabylen 30 (AcrylatesNinyl Isodecanoate-3V), Structure® 2001 polymer (Acrylates/Steareth-20 Itaconate copolymer, National Starch and Chemical Company), Structure® 3001 (Acrylates/Ceteth-20 Itaconate copolymer, National Starch and Chemical Company), Structure® Plus (Acrylates/Aminoacrylates/C10-30 Alkyl Peg 20 Itaconate copolymer, National Starch and Chemical Company), Quatrisoft® LM-200 polymer (Polyquaternium-24, available from Amerchol Corporation.), the metal oxides of titanium, zinc, iron, zirconium, silicon, manganese, aluminium and cerium, polycarbonates, polyethers, polyethylenes, polypropylenes, polyvinyl chloride, polystyrene, polyamides, polyacrylates, cyclodextrins and mixtures thereof.

Cyclodextrins are solubilized, water-soluble, uncomplexed cyclodextrins. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. Examples of preferred water-soluble cyclodextrin derivatives suitable for use herein are hydroxypropyl alpha-cyclodextrin, methylated alpha-cyclodextrin, methylated beta-cyclodextrin, hydroxyethyl beta-cyclodextrin, and hydroxypropyl beta-cyclodextrin. Cyclodextrins particularly preferred for use herein are alpha cyclodextrin, beta cyclodextrin, hydroxypropyl alpha cyclodextrin, hydroxypropyl beta cyclodextrin, and mixtures thereof.

The hair conditioner compositions are “substantially surfactant-free” and provide little or no detersive or cleaning effect on hair. Surfactants which do provide detersive effects are known in the art and include anionic, nonionic, cationic, zwitterionic, amphoteric or mixtures thereof. The substantially surfactant-free hair conditioner compositions contain less than 1% by weight of a surfactant which is to provide cleaning performance to a composition. Preferably the “substantially surfactant-free” hair conditioner compositions contains less than 0.5% by weight of a surfactant, more preferably less than 0.1% by weight of a surfactant.

For a more detailed understanding of the invention, reference can be made to the following examples which are intended as further illustration of the invention and are not to be construed in a limiting sense. All parts and percentages are by weight unless stated otherwise.

### EXAMPLE 1

Hair Conditioner Formulation: The polymers of investigation were formulated into the following formulation to evaluate for their wet and dry combing performance.

| | |
|---|---|
| 90.94 g | Deionized water |
| 00.70 g | Natrosol ® 250HHR hydroxyethylcellulose, available from |
| | Hercules Incorporated |
| 00.20 g | Polymer of this invention or commercial polymer |
| 02.00 g | Cetyl alcohol |
| 00.50 g | Potassium Chloride |
| 02.00 g | Isopropyl Myristate |
| As required | citric acid to adjust pH |
| As required | Sodium hydroxide to adjust pH |
| 00.50 g | Preservative |

The conditioner formulation was prepared by first adding Natrosol® hydroxyethyl cellulose type 250 HHR to water in a beaker and under agitation to form a slurry. Next, pH of the slurry was adjusted to 8.0 to 8.5. The slurry was stirred for about 30 minutes or until the polymer fully dissolved. Next, the polymer of this invention or a commercial polymer listed in Table 1 was added to the beaker and mixed for 30 more minutes. The solution was heated to about 65° C. and stirred until it became smooth. Next, cetyl alcohol was added to the beaker and mixed until it appeared homogeneously mixed. The mixture was cooled to about 50° C. while mixing and then potassium chloride was added. Next, isopropyl myristate was added and mixed until the mixture looked homogeneous. The pH of the mixture was adjusted between 5.25 and 5.5 with citric acid and/or NaOH solution. The conditioner was preserved with 0.5% preservative and mixing was continued until it reached room temperature. The conditioner was tested for its combing performance. All conditioners including the controls with commercial polymers contained 0.7% Natrosol® hydroxyethyl cellulose type 250 HHR. All polymers under evaluations were used at 0.2% level.

To test the conditioner for combing, flat tresses of mildly bleached European hair weighing about 3 grams were used in the study. The hair tresses were obtained from International Hair Importers and Products Inc. of Glendale, N.Y. The hair tresses were first cleaned with 4.5% active sodium lauryl sulfate solution. To clean the hair tresses, the hair tresses were first wetted with 40° C. tap water and then 5.0 ml of sodium lauryl sulfate solution was applied along each of the tresses length. Each tress was kneaded for 30 seconds. Each tress was then rinsed under 40° C. running water for 30 seconds. The hair cleaning step was repeated followed by rinsing with room temperature tap water for 30 seconds. Each tress was then dried overnight.

Next day, the sample tress was rewetted with 40° C. tap water and then 0.5 gram conditioner per gram of hair was applied uniformly along the length of hair. The tress was kneaded for 30 seconds and then it was rinsed under 40° C. running water for 30 seconds. Same amount of conditioner was reapplied again along the length of the tress and the tress was kneaded again for 30 seconds, and then it was rinsed under 40° C. running water for 30 seconds. The tress was rinsed with room temperature tap water for 30 seconds. The tress was combed immediately for eight times on an Instron instrument to measure wet combing energy (gf-mm/g). During the wet combing, the hair was sprayed with water immediately before each combing cycle to keep it wet. From the 8 combing cycle data, average wet combing energy in gram force-mm/gram of hair (gf-mm/g) was calculated. The tress was then stored overnight at about 50% relative humidity and about 23° C. For combing the tresses, an ACE rubber comb, style 61636, from Goody Products, Inc., Atlanta, Ga., was used.

Next day, the tress was first combed with fine teeth rubber comb to free-up hair stuck together. Again, the hair tress was combed eight times on an Instron instrument to determine average force required to comb one gram of dry hair. The higher the value on the Instron, the poorer the conditioning effect of the polymer being tested. Two tresses were used per hair conditioning formulation. The data reported in Table 1 are based on an average of two tresses.

TABLE 1

| | % | % | | | | |
|---|---|---|---|---|---|---|
| | Polymer | Commercial | | Conditioner | Wet | Dry |
| | of This | Cationic | Polymer | Viscosity | Combing | Combing |
| EX # | Invention | Polymer | Level | (cps) | (gf-mm/g) | (gf-mm/g) |
| Ex#1A | 0.0 | 0.0 | 0 | 990 | 4774 | 287 |
| Ex#1B | 0.0 | 0.0 | 0 | 1380 | 4513 | 364 |
| Ex#1C | | N-Hance ® 3269 | 0.2 | 1330 | 1389 | 263 |
| Ex#1D | | AguaCat ® CG518 | 0.2 | 1100 | 1142 | 332 |
| Ex#1E | EX 1 | 0.0 | 0.2 | 1930 | 889 | 124 |
| EX#1F | EX 2 | 0.0 | 0.2 | 1700 | 530 | 140 |
| Ex#1G | | UCARE ® LR400 | 0.2 | 1280 | 811 | 1116 |
| EX#1H | | UCARE ® JR30M | 0.2 | 1960 | 868 | 849 |

Ingredients used in the tests and listed in Table 1:
(1) Natrosol ® 250HHR: Hydroxyethyl cellulose from Hercules Incorporated Wilmington, DE
(2) UCARE ® LR400 HEC: Cationic HEC from Dow Chemicals, Midland, MI
(3) UCARE ® JR30M HEC: Cationic HEC from Dow Chemicals, Midland, MI
(4) N-Hance ® 3269 cationic guar, cationic DS 0.13, Weight average Molecular weight 500,000 from Hercules Incorporated, Wilmington, DE
(5) AquaCat ® CG 518: cationic guar, cationic DS 0.18, Weight average Molecular weight 50,000 from Hercules Incorporated, Wilmington, DE
(6) EX 1: Polymer of this invention, cationic guar, cationic DS 0.51: Weight average Molecular weight 1.2 MM from Hercules Incorporated. Wilmington, DE
(7) EX 2: Polymer of this invention, cationic guar cationic DS 1.06.
(8) Crodacol ™ C95NF: Cetyl alcohol from Croda Inc. Parsippany, NJ
(9) KCl: Potassium chloride
(10) Stepan ® IPM: Isopropyl myristate from Stepan Company, Northfield, IL
(11) Germaben ® II: preservative from ISP Wayne, NJ

Example 1A and 1B in Table 1 provide wet and dry combing data for the hair conditioning formulation without any type of cationic polysaccharide. The energy (gf-mm/g) required to comb wet hair is about 4500-4800. With the addition of 0.2% commercial cationic guar, Example #1C and Example #1D, the wet combing energy was about 1100-1400 gf-mm/g of hair and the dry combing energy was about the same as with the no cationic polysaccharide of Examples 1A and 1B. With the commercial cationic hydroxyethyl cellulose of Examples 1G and 1H, the wet combing energy was about 800-900 gf-mm/g of hair. However, the dry combing energy was much higher, 850-1100 gf-mm/g of hair, higher than even the conditioner without the conditioning polymer mentioned in Example 1A and 1B. The polymer of this invention not only reduced the wet combing energy to the range of 500 to 900 gf-mm/g, but also it reduced the dry combing energy to about 125-150 gf-mm/g.

### EXAMPLE 2

Hair Conditioner Formulation: The cationic guar polymers of this invention designated as EX 1, EX 2, as previously described in Table 1 and EX 3 were formulated into the following conditioner formulation to evaluate for their performance during the hair lathering, hair rinsing, wet combing and dry combing against the commercially available cationic guar

| | |
|---|---|
| 94.35 g | Deionized water |
| 01.25 g | Hydroxyethyl Cellulose (Natrosol ® 250HHR HEC |
| | available from Hercules Incorporated) |
| 00.20 g | Polymer of this invention or commercial polymer |
| 04.10 g | Stearalkonium chloride, Ammonyx ® 4002 Stepan Co. |
| 00.10 g | Preservative, methylcholoroisothiazolinone and |
| | methylisothiazolinone; Kathon ™ preservative, |
| | available from Rhom & Haas Co. |
| As required | citric acid to adjust pH |

The conditioner formulation was prepared by first adding Natrosol® hydroxyethyl cellulose type 250 HHR to water in a beaker and under agitation to form slurry. Next, pH of the slurry was adjusted to 8.0 to 8.5. The slurry was stirred for about 30 minutes or until the polymer fully dissolved. Next, the polymer of this invention or a commercial polymer listed in Table 2 was added to the beaker containing Natrosol solution and then pH was lowered to about 6 with citric acid, and the polymer solution was mixed for 30 more minutes. The solution was heated to about 50° C. in a water-bath and stirred at 50° C. for about 45 minutes. Next stearalkonium chloride (Ammonyx® LO, available from Stepan Co.) was added to the solution and allowed to mix for 30 minutes. The solution was removed from the 50° C. water-bath and cooled to room temperature while stirring. The solution pH was adjusted to about 4 to 6 with citric acid. Next, preservative was added and mixed for 15 minutes. The conditioner solution was reweighed and adjusted for ant water-loss.

Unlike the quantitative testing performed on the conditioner samples in Example 1, the conditioners were subjectively tested for their (a) Wet-Feel during the lathering/application to wet hair (b) Wet-Feel during the rinsing cycle of the hair (c) Easy of combing of wet hair (d) dry combing of hair dried overnight. Subjective ranking used was as follows. A higher the ranking, the better the product performance.

1 = No conditioning, squeaky wet feel
2 = Light conditioning
3 = Conditioning
4 = Heavy conditioning

To test the conditioner for combing, flat tresses of mildly bleached European hair weighing about 3 grams were used in the study. Two tresses were used for each formulation. The hair tresses were obtained from International Hair Importers and Products Inc. of Glendale, N.Y.

Preparation of Hair for conditioner Treatment: The hair tresses were first cleaned with 4.5% active sodium lauryl sulfate solution. To clean the hair tresses, the hair tresses were first wetted with 40° C. tap water and then 5.0 ml of sodium lauryl sulfate solution was applied along each of the tresses length. Each tress was kneaded for 30 seconds. Each tress was then rinsed under 40° C. running water for 30 seconds. The hair cleaning step was repeated followed by rinsing with room temperature tap water for 30. Each tress was then dried overnight.

Treatment with Conditioner: Next day the tress was wetted by holding vertically under running 40° C. tap water. Next, the tress was placed into a plastic weighing dish, curling in a circular manner. Then, 0.50 ml of the conditioner solution_for each gram of hair tress was applied evenly along the length of the tress with 5 ml syringe. The conditioner solution was further distributed evenly in the tress gently with the fingertips. The application took about 15 seconds. The tress was removed from the dish and held vertically. The tress was kneaded thoroughly for 30 seconds, alternately squeezing gently between the fingertips along the length of the tress, and stroking the tress to distribute the solution uniformly. The hair wet feel was noted at this lathering time for the (a) subjective evaluation. A higher the ranking better the wet feel during the lathering.

The tress was than rinsed under 40° C. running tap water while kneading as above for 30 seconds. The wet feel during rinse-off was noted at this time for the (b) subjective evaluation. A higher the ranking better the wet feel during rinse-off. Hairs were rinsed with running room temperature Deionized water for 30 seconds. After the washing with Deionized water, the excess water was squeezed out twice with the fingers.

Next hair was combed using an Ace® brand #62746 comb (coarse and fine teeth). The hair tress was combed 3 times with the coarse end of the comb, followed by 3 times with the fine end. The level of ease of combing was noted for subjective evaluation. A higher the ranking, easier the wet combing. The hair tresses were hung overnight to dry in the CT room (73° F., 50% RH). Using the fine end of an Ace® brand #62746 comb, hair tress were combed 3 times. The level of ease of combing was noted for subjective evaluation. A higher the ranking, easier the dry combing.

TABLE 2

| | % Polymer | % Commercial | Polymer | Polymer Mol | | Hair Wet Feel | | | |
|---|---|---|---|---|---|---|---|---|---|
| | of This | Cationic | Cationic DS | Wt (approx.) | Polymer | during | Hair Wet Feel | Wet | Dry |
| EX # | Invention | Polymer | (appx.) | MM | Level | lathering | during Rinsing | Combing | Combing |
| Ex#2A | — | — | 0.0 | — | 0 | 2 | 1 | 1 | 2 |
| Ex#2B | — | N-Hance ® 3215 | 0.2 | 1.2 | 0.2 | 3 | 2 | 2 | 3 |
| Ex#2C | EX 1 | — | 0.5 | 1.1 | 0.2 | 3 | 3 | 3 | 3 |
| Ex#2D | EX 2 | — | 1.1 | 1.7 | 0.2 | 3 | 3 | 3 | 4 |
| Ex#2E | EX 3 | — | 1.8 | 1.9 | 0.2 | 3.5 | 3.5 | 3.5 | 4 |

In the subjective combing test, cationic guar with higher degree of cationic substitution polymer of this invention overall provided better hair wet feel during lathering, better wet feel during rinsing cycle, easier wet combing and easier dry combing than the conditioner with commercial N-Hance® cationic guar, available from Hercules Incorporated.

### EXAMPLE 3

Hair Conditioner Formulation: The cationic guar polymers of this invention designated as EX 1, EX 2 and EX 3 were formulated into the following conditioner formulation to evaluate for their performance during the hair lathering, hair rinsing, wet combing and dry combing against the commercially available cationic guar

| | |
|---|---|
| 94.50 g | Deionized water |
| 04.50 g | Cetearyl alcohol and ceteareth-20, Promulgen ® D from |
| | Noveon Corp |
| 00.50 g | Glyceryl stearate, Emerest ® 2400 from Cognis Corp. |
| 00.20 g | PEG 5M Polyox ® WSR N-80 from Dow Chemicals |
| 00.20 g | Polymer of this invention or a commercial cationic guar |
| 00.10 g | Preservative, methylcholoroisothiazolinone and |
| | methylisothiazolinone; Kathon ® Rhom & Haas Co. |
| As required | citric acid to adjust pH |

The conditioner formulation was prepared by first adding the polymer of this invention or a commercial polymer listed in Table 3 was added to the vortex of well agitated water in a beaker. Next, pH was lowered to about 6 with citric acid, and the polymer solution was mixed for 30 more minutes. The solution was heated to about 75° C. in a water-bath and stirred at 75° C. This was a water-phase. In a separate beaker, oil phase prepared by melting glyceryl stearate, PEG and cetearyl alcohol and ceteareth-20 at 75° C. Next, the oil phase was slowly added to the water phase under agitation at 75° C. Mixing was continued for 10 minutes. The emulsion was removed from the water-bath and cooled to room temperature while mixing. The emulsion pH was adjusted to about 5 with citric acid. Next, the preservative was added and mixed for 15 minutes. The conditioner solution was reweighed and adjusted for water-loss.

The conditioners were subjectively tested for their (a) Wet-Feel during the lathering/application to wet hair (b) Wet-Feel during the rinsing cycle of the hair (c) Easy of combing of wet hair (d) dry combing of hair dried overnight. Subjective ranking used was as follows. A higher the ranking, the better the product performance.

1 = No conditioning, squeaky wet feel
2 = Light conditioning
3 = Conditioning
4 = Heavy conditioning

To test the conditioner for combing, flat tresses of mildly bleached European hair weighing about 3 grams were used in the study. Two tresses were used for each formulation. The hair tresses were obtained from International Hair Importers and Products Inc. of Glendale, N.Y. The hair tress evaluation was conducted using the method described in Example 2.

TABLE 3

| | % Polymer | % Commercial | Polymer | Polymer Mol | | Hair Wet Feel | | | |
|---|---|---|---|---|---|---|---|---|---|
| | of This | Cationic | Cationic DS | Wt (approx.) | Polymer | during | Hair Wet Feel | Wet | Dry |
| EX # | Invention | Polymer | (appx.) | MM | Level | lathering | during Rinsing | Combing | Combing |
| Ex#3A | — | — | 0.0 | — | 0 | 1 | 1 | 1 | 1 |
| Ex#3B | — | N-Hance ® 3215 | 0.2 | 1.2 | 0.2 | 3 | 3 | 3 | 4 |
| Ex#3C | EX 1 | — | 0.5 | 1.1 | 0.2 | 3 | 3 | 3 | 4 |
| Ex#3D | EX 2 | — | 1.1 | 1.7 | 0.2 | 3 | 3 | 3 | 4 |
| Ex#3E | EX 3 | — | 1.8 | 1.9 | 0.2 | 3 | 3 | 3 | 4 |

In our subjective combing test, cationic guar based formulation prepared with higher cationic degree of substitution polymer of this invention or with the commercial cationic guar performed equal but better than a conditioner with no cationic guar polymer.

### EXAMPLE 4

Hair Conditioner Formulation: The cationic guar polymers of this invention designated as EX 1, EX 2 and EX 3 were formulated into the following conditioner formulation to evaluate for their performance during the hair lathering, hair rinsing, wet combing and dry combing against the commercially available cationic guar.

| | |
|---|---|
| 94.35 g | Deionized water |
| 01.50 g | Hydroxyethylcellulose, Natrosol ® 250HHR from |
| | Hercules, Inc. |
| 00.20 g | Tetrasodium EDTA, Versene ® 220 from Dow Chemicals. |
| 00.30 g | Cetrimonium chloride Ammonyx CETAC from Stepan Co. |
| 01.00 g | Cetearyl alcohol, Crodacol ® CS50, from Croda |
| 01.00 g | Glyceryl stearate and PET 100 stearate, Arlacel ® from |
| | Uniqema. |
| 02.00 g | Silicone emulsion, Dow Corning 1784 from Dow Corning |
| 00.60 g | Polymer of this invention or a commercial cationic guar |
| 00.40 g | Preservative, DM DM Hydantoin, Glydant from Lonza. |
| As required | citric acid to adjust pH |

The conditioner formulation was prepared by first adding Natrosol® hydroxyethyl cellulose type 250 HHR to water in a beaker and under agitation to form slurry. Next, pH of the slurry was adjusted to 8.0 to 8.5. The slurry was stirred for about 30 minutes or until the polymer fully dissolved. Next, the polymer of this invention or a commercial polymer listed in Table 4 was added to the beaker containing Natrosol solution and then pH was lowered to about 6 with citric acid, and the polymer solution was mixed for 30 more minutes. The solution was heated to about 75° C. in a water-bath and stirred at 75° C. Next, tetrasodium EDTA, cetrimonium chloride, cetearyl alcohol, glyceryl stearate and PET 100 stearate were added. Between each addition, the formulation was mixed for 10 minutes. The solution was removed from the 75° C. water-bath and cooled to room temperature while stirring. Next, Silicone emulsion was added and followed by preservative. The solution pH was adjusted to about 5 with citric acid. The conditioner solution was reweighed and adjusted for any water-loss.

The conditioners were subjectively tested for their (a) Wet-Feel during the lathering/application to wet hair (b) Wet-Feel during the rinsing cycle of the hair (c) Easy of combing of wet hair (d) dry combing of hair dried overnight. Subjective ranking used was as follows. A higher the ranking, the better the product performance.

1 = No conditioning, squeaky wet feel
2 = Light conditioning
3 = Conditioning
4 = Heavy conditioning

To test the conditioner for combing, flat tresses of mildly bleached European hair weighing about 3 grams were used in the study. Two tresses were used for each formulation. The hair tresses were obtained from International Hair Importers and Products Inc. of Glendale, N.Y. The hair tress evaluation was conducted using the method described in example 2.

TABLE 4

| | | | | Polymer | | | | | |
|---|---|---|---|---|---|---|---|---|---|
| | Polymer | | Polymer | Mol Wt | | Hair Wet | Hair Wet | | |
| | of This | Comm. Cationic | Cationic DS | MM | Polymer | Feel | Feel | Wet | Dry |
| EX # | Invention | Polymer | approx | (approx) | Level | during lathering | during Rinsing | Combing | Combing |
| Ex#4A | — | — | 0.0 | — | 0 | 2 | 2 | 2 | 3 |
| Ex#4B | — | N-Hance ® 3215 | 0.2 | 1.2 | 0.6 | 2 | 3 | 3 | 4 |
| Ex#4C | EX 1 | — | 0.5 | 1.1 | 0.6 | 3 | 3 | 3 | 4 |
| Ex#4D | EX 2 | — | 1.1 | 1.7 | 0.6 | 3 | 3 | 3 | 4 |
| Ex#4E | EX 3 | — | 1.8 | 1.9 | 0.6 | 3. | 3 | 3. | 4 |

In subjective combing test, cationic guar based formulation prepared with higher cationic degree of substitution polymer of this invention a very slight improvement hair wet feel during the lathering was observed over the commercial cationic guar.

Although the invention has been described with referenced to preferred embodiments, it is to be understood that variations and modifications in form and detail thereof may be made without departing from the spirit and scope of the claimed invention. Such variations and modifications are to be considered within the purview and scope of the claims appended hereto.

What is claimed:

1. A hair conditioner composition consisting essentially of:
a) from about 1 to about 99 wt % of water;
b) a lower limit amount of 0.05 wt % of a cationic polymer that has
i) a weight average molecular weight (Mw) of from about 2,000 to about 10,000,000 Dalton, and
ii) a cationic degree of substitution (DS) with a lower limit of 0.25 and an upper limit of 3.0; and
c) at least one hair conditioner active ingredient,
wherein the hair conditioner composition is substantially surfactant-free.

2. The hair conditioner composition of claim 1, wherein the active hair conditioner ingredient is selected from the group consisting of perfumes, emollients, moisturizing agents, humectants, sunscreen actives, beauty aids, anti-oxidant agent, antimicrobial agent, additional conditioning agent, depositing agent, tanning agent, rejuvenating agent, medicaments, colorant, anti-static agent, and mixtures thereof.

3. The hair conditioner composition of claim 1, wherein the cationic polymer is present in the composition with the lower limit amount of 0.1 wt %.

4. The hair conditioner composition of claim 1, wherein the cationic polymer is present in the composition with the lower limit amount of 0.5 wt %.

5. The hair conditioner composition of claim 1, wherein the cationic polymer is present in the composition with the lower limit amount of 1.0 wt %.

6. The hair conditioner composition of claim 1, wherein the cationic polymer is present in the composition with the upper limit amount of 10.0 wt %.

7. The hair conditioner composition of claim 1, wherein the cationic polymer is present in the composition with the upper limit amount of 5.0 wt %.

8. The hair conditioner composition of claim 1, wherein the cationic polymer is present in the composition with the upper limit amount of 3.0 wt %.

9. The hair conditioner composition of claim 1, wherein the composition has a cationic degree of substitution (DS) lower limit of 0.4.

10. The hair conditioner composition of claim 1, wherein the cationic degree of substitution (DS) has a lower limit amount of 0.8.

11. The hair conditioner composition of claim 1, wherein the cationic degree of substitution (DS) has an upper limit amount of 2.0.

12. The hair conditioner composition of claim 1, wherein the cationic degree of substitution (DS) has an upper limit amount of 1.0.

13. The hair conditioner composition of claim 1, wherein the cationic polymer is at least one cationic polygalactomannan or cationic derivatized polygalactomannan.

14. The hair conditioner composition of claim 13, wherein the derivative moiety on the cationic derivatized polygalactomannan is selected from the group consisting of alkyl, hydroxyalkyl, alkylhydroxyalkyl, and carboxymethyl wherein the alkyl has a carbon chain containing from 1 to 30 carbons and the hydroxyalkyl is selected from the group consisting of hydroxyethyl, hydroxypropyl, hydroxybutyl and mixtures thereof.

15. The hair conditioner composition of claim 13, wherein the polygalactomannan is selected from the group consisting of guar, locust bean, honey locus, and flame tree.

16. The hair conditioner composition of claim 13, wherein the cationic moiety is selected from quaternary ammonium compounds.

17. The hair conditioner composition of claim 16, wherein the quaternary ammonium compound is selected from the group consisting of 3-chloro-2-hydroxypropyltrimethylammonium chloride, 2,3-epoxy-propyltrimethylammonium chloride, 3-chloro-2-hydroxypropyltrimethylammonium bromide, 2,3-epoxy-propyltrimethylammonium bromide; glycidyltrimethylammonium chloride, glycidyltriethylammonium chloride, gylcidyltripropylammonium chloride, glycidylethyldimethylammonium chloride, glycidyldiethylmethylammonium chloride, and their corresponding bromides and iodides; 3-chloro-2-hydroxypropyltrimethylammonium chloride, 3-chloro-2-hydroxypropyltriethylammonium chloride, 3-chloro-2-hydroxypropyltripropylammonium chloride, 3-chloro-2-hydroxypropylethyldimethylammonium chloride, and their corresponding bromides and iodides; and halides of imidazoline ring containing compounds.

18. The hair conditioner composition of claim 1, wherein the Mw has a lower limit of 10,000 Dalton.

19. The hair conditioner composition of claim 1, wherein the Mw has a lower limit of 50,000 Dalton.

20. The hair conditioner composition of claim 1, wherein the Mw has a lower limit of 100,000 Dalton.

21. The hair conditioner composition of claim 1, wherein the Mw has a lower limit of 400,000 Dalton.

22. The hair conditioner composition of claim 1, wherein the Mw has an upper limit of 5,000,000 Dalton.

23. The hair conditioner composition of claim 1, wherein the Mw has an upper limit of 2,000,000 Dalton.

24. The hair conditioner composition of claim 1, wherein the Mw has an upper limit of 1,000,000 Dalton.

25. The hair conditioner composition of claim 1, further consisting essentially of a member selected from the group consisting of colorant, preservative, antioxidant, activity enhancer, emulsifier, functional polymer, viscosifying agent, alcohol, fat or fatty compound, antimicrobial compound, silicone material, hydrocarbon polymer, oil, vitamins, suspending agents, stabilizing biocide, pH modifier, flavor, fragrance, salt, and mixtures thereof.

26. The hair conditioner composition of claim 25, wherein the functional polymer is selected from the group consisting of anionic, hydrophobically-modified, and amphoteric acrylic acid copolymers, vinylpyrrolidone homopolymers and copolymers, cationic vinylpyrrolidone copolymers, nonionic, cationic, anionic, and amphoteric cellulosic polymers, acrylamide homopolymers, cationic, anionic, amphoteric, and hydrophobically-modified acrylamide copolymer, polyethylene glycol polymer and copolymer, hydrophobically-modified polyether, hydrophobically-modified polyetheracetal, hydrophobically-modified polyetherurethane, an associative polymer, hydrophobically-modified cellulosic polymer, polyethyleneoxide-propylene oxide copolymer, and a nonionic, anionic, hydrophobically-modified, amphoteric, and cationic polysaccharides, chitosan, and mixtures thereof.

27. The hair conditioner composition of claim 26, wherein the nonionic, cationic, anionic, and amphoteric cellulosic polymers are selected from the group consisting of hydroxyethylcellulose, hydroxypropylcellulose, hydroxypropylmethylcellulose, methylcellulose, carboxymethylcellulose, hydrophobically-modified carboxymethylcellulose, carboxymethyl hydroxyethylcellulose, cationic hydroxyethylcellulose, ethylhydoxyethylcellulose, cationic hydrophobically-modified hydroxyethyl cellulose, hydrophobically modified hydroxyethylcellulose, hydrophobically-modified hydroxypropylcellulose, cationic hydrophobically-modified hydroxypropyl cellulose, cationic carboxymethylhydroxyethylcellulose, and cationic hydroxypropylcellulose.

28. The hair conditioner composition of claim 26, wherein the nonionic, anionic, hydrophobically modified, amphoteric, and cationic polysaccharides are selected from the group consisting of carboxymethyl guar, alginates, hydroxypropyl guar, hydrophobically-modified guar, carboxymethyl guar, hydroxypropyl carboxymethyl guar, hydroxyethyl guar, hydroxypropyltrimethylammonium chloride, guar hydroxypropyltrimethylammonium chloride, and hydroxypropyl guar hydroxypropyltrimethylammonium chloride.

29. The hair conditioner composition of claim 25, wherein the viscosifying agent is selected from the group consisting of NaCl, NH₄Cl, KCl, Na₂SO₄, fatty alcohols, fatty acid esters, fatty acid amides, fatty alcohol polyethyleneglycol ethers, sorbitol polyethyleneglycol ethers, cocamidopropyl betaine, clays, silicas, cellulosic polymers, xanthan, and mixtures thereof.

30. The hair conditioner composition of claim 25, wherein the silicone material is selected from the group consisting of cyclosiloxane, linear siloxane, siloxane structure with polyol, amino, or other functional groups in the siloxane structure, and mixtures thereof.

31. The hair conditioner composition of claim 30, wherein the other functional groups are selected from the group consisting of polyethyleneoxy and/or polypropyleneoxy groups optionally containing C₆-C₂₄ alkyl groups, substituted or unsubstituted amine groups, thiol groups, alkoxylated groups, hydroxyl groups, acyloxyalkyl groups.

32. The hair conditioner composition of claim 25, wherein the silicone material is selected from the group consisting of polyalkylsiloxanes, polyarylsiloxanes, polyalkylarylsiloxanes, and mixtures thereof.

33. The hair conditioner composition of claim 32, wherein the polyalkylsiloxanes are selected from the group consisting of polydimethylsiloxane, polydimethylsiloxane hydroxylated at the end of the chain, and mixtures thereof.

34. The hair conditioner composition of claim 1, wherein the hair conditioner composition is selected from the group consisting of leave-on hair conditioner, leave-in conditioner, rejuvenating conditioner, creme rinse, oil-free hair conditioners, rinse-off hair conditioner, conditioning rinse, foaming conditioner, conditioning styling gel, conditioning mousse, spay-on conditioner, hair dressing crème, hair repair spray, and mixtures thereof.

35. The hair conditioner composition of claim 2, wherein additional conditioning agent is selected from the group consisting of silicone materials, hydrocarbon oils, vegetable oils, vegetable fats, animal oils, animal fats, fatty acid, fatty alcohol, fatty esters, panthenol and derivatives thereof, pantothenic acid and derivatives thereof, and mixtures thereof.

36. The hair conditioner composition of claim 1, wherein the hair conditioner composition is an oil-in-water or water-in-oil emulsion or solution or dispersion or suspension.
